(19) United States
(12) Patent Application Publication
(10) Pub. No.: US 2004/0121943 A1
(43) Pub. Date: Jun. 24, 2004

(54) Drug-free biodegradable 3D porous collagen-glycosaminoglycan scaffold

(76) Inventors: Wei-Cherng Hsu, Taipei (TW); Jo-Yi Hsiao, Taoyuan (TW); Hsiao-Cheng Yen, Taichung (TW)

Correspondence Address: Jason Z. Lin, Supreme Patent Services, Post Office Box 2339, Saratoga, CA, 95070, US

(21) Appl. No.: 10/327,528

(22) Filed: Dec. 20, 2002

(51) Int. Cl. A61K 38/38

(52) U.S. Cl. 514/8; 530/356

(57) ABSTRACT

A drug-free biodegradable 3D porous collagen-glycosaminoglycan scaffold is designed for preventing scar formation and creating a physiological aqua buffer environment around the conjuctival space for glaucoma. The scaffold improves the re-modeling of the regenerating tissue and prevents scar formation and further infection. It is prepared without further chemical linkage, and consequently becomes softer and without the uncertainty of chemical remnants after implantation. In addition, the scaffold can be cut preferably in a form upon request before being saturated by a physiological buffer for implantation.

### FIELD OF THE INVENTION

The present invention generally relates to a drug-free biodegradable 3-dimentioned porous collagen-glycosaminoglycan scaffold serving as an implantation device, and in particular to a device designed for preventing scar formation and creating a physiological aqua buffer environment in conjunctival space for modulating the intraocular pressure of glaucoma.

### BACKGROUND OF THE INVENTION

Glaucoma encompasses serial symptoms such as intraocular pressure elevation, optic nerve damage and progressive visual field loss. Most patients receive medical treatments by oral ingestion or locally applying beta-blockers, miotics, adrenergic agonists or carbonic anhydrase inhibitors to enhance water reabsorption by blood vessels and consequently lower the intraocular pressure. Most of the patients significantly respond to drug therapy at the beginning, but many cases turn out to be refractory over time. For the individual who fails to quickly respond to drug treatment, surgical intervention is required in order to maintain intraocular pressure. Glaucoma filtering surgery is the current operating process for reducing intraocular pressure. The processes of glaucoma filtering surgery consist of making an opening through the trabeculum to drain out aqueous humor from the anterior chamber, and building a filtering bleb or drainage fistula between the anterior chamber and the subconjuctival space to reduce intraocular pressure (Bergstrom et al., 1991; Miller et al., 1989). However, the scar development after surgery results in the obstruction of the built filtering bleb or drainage fistula and finally leads to the recurrence of high intraocular pressure (Peiffer el al., 1989). Hence, the prevention of scar formation should be the most important consideration for the success of glaucoma surgery.

Clinical treatments use mitomycin-C, 5-fluorouracil, bleomycin, β-aminopropionitrile, D-penicillamine, tissue plasminogen activator and corticosteroid for the inhibition of fibroblast proliferation to prevent scar development after glaucoma surgery. Nevertheless, observed side effects, such as thinning of the conjunctiva or intraorbital inflammation can lead to blindness.

Prior arts U.S. Pat. No. 5,713,844 and U.S. Pat. No. 5,743,868 disclosed the pump- or tube-like devices made with artificial materials being implanted into the subconjunctival space or the anterior chamber surroundings as an alternative to the filtering bleb or drainage fistula to lower the intraocular pressure. These non-degradable devices function as the fistula and bleb, giving short-term benefits but the procedure eventually fails due to scar formation. Moreover, the devices are not biodegradable, causing incommodity and risk of secondary infection. In addition, no clinical observation shows significant reduction of scar formation after implanting such devices. As a matter of fact, the regenerative tissue often invades or pinches into the implanted devices, consequently obstructing the outflow pathway. For the most part, it is not a general therapeutic consideration.

For years, studies on tissue engineering achieved great progress in scar prevention (Yannas et al., 1989; Yannas, 1998). For example, artificial skin contributes great benefits to wound healing (Orgill et al., 1996; Yannas et al., 1982). U.S. Pat. No. 4,060,081 and U.S. Pat. No. 5,489,304 disclosed artificial skin to benefit wound healing and prevent scar formation. Both types of artificial skin combine a degradable layer and another non-degradable layer. The non-degradable layer composed of synthetic polymers controls moisture flux of the skin; and the degradable layer composed of a three-dimensioned (3D) collagen-mucopolysaccharide or collagen-glycosaminoglycan copolymer directly covers the wound area to support tissue regeneration. The 3D collagen-mucopolysaccharide or collagen-glycosaminoglycan copolymers lead a random reorganization of the regenerating fibroblasts and the secreted intercellular matrix, and finally result in a reduction of scar formation. To mimic skin physiological function, the prior arts have been designed with a high intensity of chemical linkage between components and functional control of the moisture flux. In addition, these products are generally for external application, rather than for use as an implanting device. It is not possible to apply such artificial skin as an implanting device directly in a glaucoma treatment. Another resolution for preventing scar formation and modulating intraocular pressure after glaucoma surgery is highly desirable.

U.S. Pat. No. 6,299,895 and U.S. Pat. No. 6,063,116 disclosed implanting devices, which carried different biological active molecules to inhibit cell proliferation, amend tissue regeneration and prevent scar development. However, the building components are not fully biodegradable. U.S. Pat. No. 6,013,628 and U.S. Pat. No. 6,218,360 presented a combination of cell proliferating inhibitors and different biodegradable mediators, and the direct application into the intraocular tissue. Although these patents solved the problem of the non-degradability of the drug mediator, there is still the risk that the drug may leak out from the injecting site. The affected area will be beyond control. Moreover, the probability of repetitional injection is often required.

### SUMMARY OF THE INVENTION

The present invention provides a 3D porous collagen-glucosaminoglycans scaffold, which is fully biodegradable after being implanted into the subconjuctival space. The 3D porous structure reduces intraocular pressure, leads a re-arrangement of proliferating cells and matrix, prevents scar formation, and provides a permanent physiological aqua reservoir system after biodegrading.

An object of the invention is to provide a new device for glaucoma implantation. In some preferred embodiments, there are provided methods of purifying type I collagen and making a biodegradable 3D porous collagen/glucosaminoglycan scaffold serving as an implanting device. The device leads to cell re-organization during regeneration and builds a physiological aqua buffer reservoir for the modulation of intraocular pressure after glaucoma surgery. On the other hand, no further aldehyde linkage has been conducted during preparation procedures, and consequently reduces the hardness and the risk of chemical remnants.

A further object of the invention is to provide a special procedure of implanting the device into animals' subconjunctival space. No drug should be added during and after the implantation. The present invention prevents scar development and modulates the intraocular pressure based only on the 3D porous structure and the biodegraded residual space. The present invention is not used as a drug mediator or drug carrier.

In one embodiment, the intraocular pressure has been measured after implantation. In other embodiments, different cellular evaluations were also performed on the days 3, 7, 14, 21 and 28 after implantation, so as to monitor the scaffold biodegradation and the tissue regeneration.

The foregoing and other objects, features, aspects and advantages of the present invention will become better understood from a careful reading of a detailed description provided herein below with appropriate reference to the accompanying drawings.

### BRIEF DESCRIPTION OF THE DRAWINGS

FIG. 1 shows the change of the static pressure of scaffolds in different concentrations of collagen-glycosaminoglycan.

FIG. 2 shows the morphological evaluation after scaffold implantation in female New Zealand albino rabbits. Wherein (a)(c)(e)(g) shows the results from the implanted groups, and (b)(d)(f)(h) from the operating sham groups.

(a) The immune-responded cells infiltrated into the cross-referred area of the implanted area (*). The scaffold was degraded partially and some regenerated cells invaded this area (↑). (H&E stain, 40x, Day 3).

(b) The immune-responded cells infiltrated into the cross-referred area of the operating sham groups (*). (H&E stain, 40x, Day 3).

(c) Identified fibroblasts (▴) and secreted collagen (↑) randomly arranged in the cross-referred area of the implanted area. (Masson Trichrome stain, 400x, Day 14).

(d) Identified fibroblasts (▴) and secreted collagen (↑) compactly arranged in the cross-referred area of the operating sham groups. (Masson Trichrome stain, 400x, Day 14).

(e) Very few α-SMA immuoreactive cells (↑) randomly appeared in the remaining area of degraded scaffold. (α-SMA immunocytochemistry, 400x, Day 14).

(f) Numerous a -SMA immuoreactive cells (↑) compactly arranged in the cross-referred area of the operating sham groups. (α-SMA immunocytochemistry, 400x, Day 14).

(g) Very little identified collagen randomly distributed in the remaining area of fully degraded scaffold (↑) . (Masson Trichrome stain, 2x, Day 28).

(h) Typical scar tissue (↑) shown as compactly arranged collagen fibers distributed in the cross-referred area of the operating sham groups. (Masson Trichrome stain, 2x, Day 28).

FIG. 3 indicates the development of the intraocular pressure after scaffold implantation.

### DETAILED DESCRIPTION OF THE PREFERRED EMBODIMENTS

The present invention provides a fully biodegradable 3D porous scaffold, which is comprised of collagen-glucosaminoglycans copolymers. Although numerous studies and patents described the use of collagen alone or in combination with other components as biomaterials, the present invention sets high temperature (see examples) and UV light as the major energy for polymerization, and non-obviously, no further aldehyde linkage reaction has been done through the preparation. Hence, there are no aldehyde remnants. As a result, the final product not only maintains the 3D porous structure to lead the regenerating tissue reorganization but also is softer in comparison to those disclosed in other prior arts (U.S. Pat. No. 5,629,191, U.S. Pat. No. 6,063,396, and Hsu et al., 2000).

On the other hand, many prior arts provide implanting devices to be drug mediators or carriers, wherein the drugs released from mediators or carriers locally inhibit cell proliferation and prevent scar development. However, the drug re-filling is complicated and no side effects have been evaluated for certain drugs. The present invention thus offers a drug-free biodegradable 3D porous scaffold as the resolution of these issues. The scaffold prevents scar formation by directly leading the proliferating cells and matrix to scattered rearranging in its 3D porous structure. Consequently, the residual space after the scaffold being degraded is filled with loose connective tissue, and works as a permanent water reservoir to buffer intraocular pressure. The scaffold not only solves the recurrence of abnormal intraocular pressure but also eliminates the risk which might occur during drug loading and its side effects.

The comprising ratio of collagne-glycosaminoglycan copolymers for the scaffold used as a glaucoma implant ranges from 0.125% to 8%, wherein the collagen is type I collagen and the glycosaminoglycan is comprised of chondroitin-6-sulfate, chondrotin-4-sulfate, heparin, heparan sulfate, keratan sulfate, dermatan sulfate, chitin and/or chitosan. Type I collagen and the different glycosaminoglycans crosslink in the ratio of 10:1 by weight through high temperature and being thoroughly mixed at a high speed. To maintain the scaffolds being softer than those being fabricated with aldehyde linkage after being saturated with physiological phosphate buffered saline (PBS), there is no secondary aldehyde linkage during the preparation. Preferably, the scaffold should be kept dry until it is prepared for implantation.

The present invention applies to glaucoma surgery. Appropriate collagen/glycosaminoglycan copolymers containing the ratio and size of the disclosed scaffold have been cut and saturated with physiological phosphate buffered saline. Carefully dissect the conjunctiva from the fornix to the limbus, and expose the sciera. Make a trabecular channel connecting the subconjunctival space and the anterior chamber. Implant the PBS saturated scaffold into the subconjuctival space surrounding and above the sclera flap, and including the trabecular channel if necessary. The PBS saturated scaffold provides a static pressure against the intraocular pressure to avoid excessive aqueous humor leaking out from the anterior chamber, and consequently prevents hypotony shortly after glaucoma surgery. The biodegradable 3D porous structure of the implanted scaffold provides a drug-free and chemical-free environment to lead to the rearrangement of the proliferating cells and matrix, and finally to prevent scar development. It results in a loose tissue structure after fully degrading. The loose structure then offers a permanently physiological aqueous humor buffer reservoir to modulate intraocular pressure.

The following examples are shown in the way of illustration instead of limitation.

### EXAMPLE 1

### Preparation of Type I Collagen

Three hundred grams of bovine tendon is chopped into small pieces of about 0.5 cm³ and mixed with 10 liters of 95% ethanol at 4° C. for 24 hours. Transfer the tendon pieces into 10 liters of 0.5 M acetic acid solution and stir the mixture at 4° C. for 72 hours. Add pepsin (SIGMA P7000, 4000 unit/ml) to the mixture and stir the mixture at 4° C. for 24 hours. Filter the mixture and discard the remnants. Add sodium chloride to the solution and adjust the final concentration to 1.0 M. Mix the solution under magnetic stirring at 4° C. for 30 minutes. Centrifuge the prepared solution at 10,000 g (Beckman Avanti J-20) for 30 minutes and remove the supernatant. Resuspend the pellet by adding 10 liters of 50 mM Tris-HCI solution (pH7.4) and stir the solution at 4° C. for 30 minutes. Add sodium chloride to a final concentration of 4.0 M. Mix the solution completely at 4° C. for 30 minutes. Remove the supernatant after being centrifuged at 10,000 g for 30 minutes. Resuspend the pellet with 10 liters of 50 mM Tris-HCI solution (pH7.4), and mix the solution completely at 4° C. for 30 minutes. Add sodium chloride again to the solution until the final concentration is 2.5 M, and stir the solution for 30 minutes at 4° C. Remove the supernatant after being centrifuged at 10,000 g for 30 minutes. Add 5 liters of mixed solution of isopropanol and H₂O (Isopropanol: H₂O=1:4) to resuspend the pellet, and mix at 4° C. for 30 minutes under magnetic stirring. Remove the supernatant after being centrifuged at 10,000 g at 4° C. for 30 minutes, and resuspend the pellet with 5 liters of 0.05 M acetic acid solution. Repeat the procedure of centrifugation/resuspension twice. Freeze the solution at −90° C. Lyophilize the solution and obtain the desiccated product of Type I collagen.

Preparation of durg-free biodegradable 3D porous collagen/glucosaminoglycan scaffold

Dissolve 4.8 g of type I collagen, obtained from Example 1, in 400 ml of 0.05 M acetic acid. Mix the solution in a water bath at 10° C. under magnetic stirring stepwise from 3,500 rpm for 60 minutes, 7,000 rpm for 30 minutes to 11,500 rpm for 60 minutes. Dissolve 0.48 g of chondroitin-6-sulfate (C-6-S) in 80 ml of 0.05 M acetic acids. Then mix the C-6-S solution with type I collagen solution under magnetic stirring stepwise from 3,500 rpm for 60 minutes, 7,000 rpm for 30 minutes to 11,500 rpm for 60 minutes. Pour the collagen and C-6-S mixture into a 4-liter flask. Vacuum the mixture until the pressure is lower than 30 mtorr and store the mixture at 4° C. Place 160 ml of the cold collagen and C-6-S mixture in a 14 cm ×22 cm stainless tray. Lyophilize the collagen and C-6-S mixture at −90° C., until a sheet-like collagen and C-6-S mixture has been obtained. Seal the sheet of collagen and C-6-S mixture in an aluminum-foil bag and polymerize the collagen and C-6-S mixture by exposure to a vacuum at a temperature of 105° C. for 24 hours. Take out the sheets of collagen/C-6-S copolymer from the aluminum-foil bag, and further crosslink by exposure to 254 nm UV for 2 hours each side in a UV crosslinker. Store the 3D porous sheet of collagen/C-6-S copolymer in a dry aluminum-foil bag at 4° C.

The ratio of collagen/glycosamnioglycans on the scaffold is maintained at 10:1. However, the contents of the collagen/glycosamnioglycan copolymer can be changed in a preferred range of 0.125%-8%. The obvious difference between the present invention and those disclosed in prior arts is that no further aldehyde cross-linkage has been applied during the scaffold preparation. Therefore, there is no risk of chemical remnants. In addition, the obtained scaffold is much softer, since no secondary chemical cross-linkage has been done during the preparation.

### EXAMPLE 2

### Measurement of the Static Pressure after being Saturated by Physiological Buffer

The drug-free biodegradable 3D porous scaffold containing 0.25%, 0.5% and 1% collagen/C-6-S copolymers separately are cut into discs with 7, 7.5, 8, 8.5, and 9 mm in diameter and 2 to 3 mm in thickness. Weigh the discs by a scale and take records. Place the discs in 0.1 M PBS until the collagen/C-6-S copolymers are saturated and weigh the discs. Repeat the steps 10 times. Calculate the saturated static pressure of the scaffold per unit area on the basis of the following equation. Variation of the measurements is evaluated by a t-test.

Saturated static pressure of the scaffold (mmHg)=[Weight of the saturated scaffold (mg)—Weight of the dry scaffold (mg)]×0.0736/ Area of the scaffold (mm²)

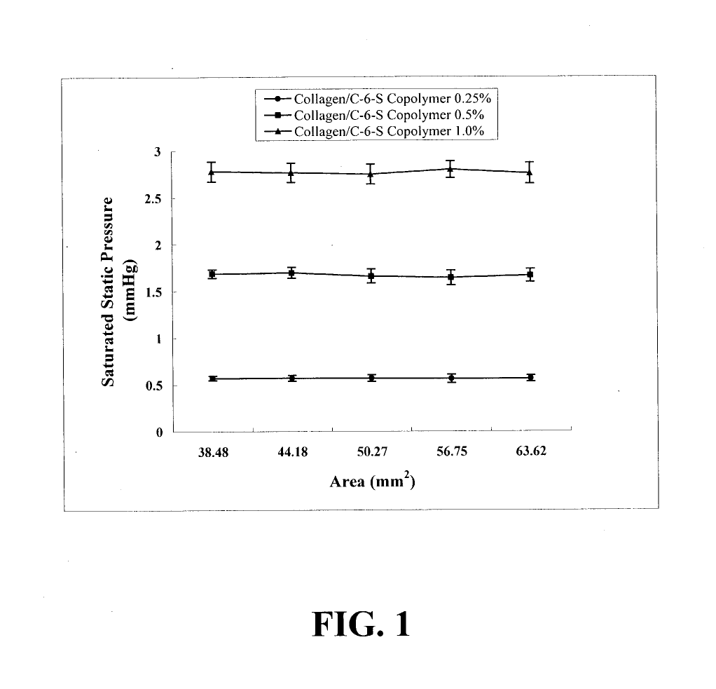

The saturated static pressure of the scaffold is the maximum anticipating intraocular buffering pressure. The data indicates that the greater the concentration of collagen/C-6-S copolymer in the scaffold is, the greater the saturated static pressure increases (see FIG. 1). This is because collagen molecules have high affinities of binding with H₂O. In addition, the data shows that the scaffold with the same concentration of collagen/C-6-S copolymers but different in size has a property where the saturated static pressure is in direct proportion to the size of the area. The result indicates the stable and homogeneous on nature of collagen/C-6-S copolymers. Hence, the scaffold with various concentrations of collagen/C-6-S copolymers and different shapes can be prepared in advance upon different demands.

### EXAMPLE 3

### Animal Model of the Implantation of the Drug-free Biodegradable 3D Porous Scaffold in Regulating the Intraocular Pressure on Glaucoma

The drug-free biodegradable 3D porous scaffold of 0.5% collagen/C-6-S copolymer is cut into several identical small discs of 8-mm in diameter and 2-3 mm in thickness. The discs are immersed exhaustively in 0.1 M PBS for 4 to 6 hours to be saturated. Seventeen female New Zealand albino rabbits weighing between 2.5 to 3.5 kg are anesthetized by an intramuscular injection of ketamine (35 mg/kg, BW) and xylazine (5 mg/kg, BW). All the scaffolds are implanted in the animals' right eyes with their left eyes serving as the surgical sham control. Open the eyelids with a speculum. A wound of approximately 8-10 mm in length is made by ophthalmic scissors on the right eye. The wound is located between the 10 o'clock and 12 o'clock position at a distance of 2 mm away from the corneal-scleral limbus. Separate the conjunctival epithelium and substantia propia to expose the sclera. Build a channel over the trabeculum to connect the anterior chamber and subconjuctival space, wherein implant the scaffold. Seal the wound. To be a surgical sham control, the same surgical procedures are done on the left eyes without the scaffold implantation.

### EXAMPLE 4

### Histological Evaluation After the Drug-free Biodegradable 3D Porous Scaffold Implantation

Totally, 17 implanted rabbits are sacrificed by excess anesthetics of ketamine (2×35 mg/kg BW ) and xylazine (2×5 mg/kg, BW ) on day 3, 7, 14, 21, and 28 after implantation. Quickly remove the eyes including the eyelids and fix them in 4% formaldehyde overnight. The implant and underlying scleral bed is dissected, dehydrated, and embedded in paraffin. Sections are cut by a microtome at 7 μm and stained with H&E (hematoxylin and eosin) for general histological observation, and Masson trichrome stain to assess collagen deposition and remodeling. Additional tissue sections are used for the α-SMA (α-smooth muscle actin) immunocytochemistry to identify the distribution of myofibroblasts. The procedures of H&E stain, Masson's trichrome stain, and α-SMA immunocytochemistry are described below:

Evaluation of the General Histology by H&E Stain After Implanting the Drug-free Biodegradable 3D Porous Scaffold:

Deparaffin the tissue sections by heating the slides in 56° C. for 10 minutes and immersing in 100% xylene for 3 minutes (repeat 3 times). Transfer the slides in 100% ethanol for 2 minutes (repeat 3 times) and rehydrate sequentially to 90%, 80%, 70%, and 50% ethanol for 3 minutes each step. Stain the slides in hematoxylin solution for 10 minutes and remove the excessive dye in distilled water for 5 minutes (repeat 2 times). Then place the slides in eosin solution for 20 seconds. Wash the slides in distilled water to remove the excessive dye for 5 minutes (repeat 2 times). The stained tissue is dehydrated by sequential 50%, 70%, 80%, 90%, 100% ethanol for 10 seconds each. After the secondary treatment in 100% ethanol, place the slides in the 100% xylene for 10 seconds (repeat 3 times). Cover the slides with Permount or Polymount, and observe under light microscopy.

RESULTS

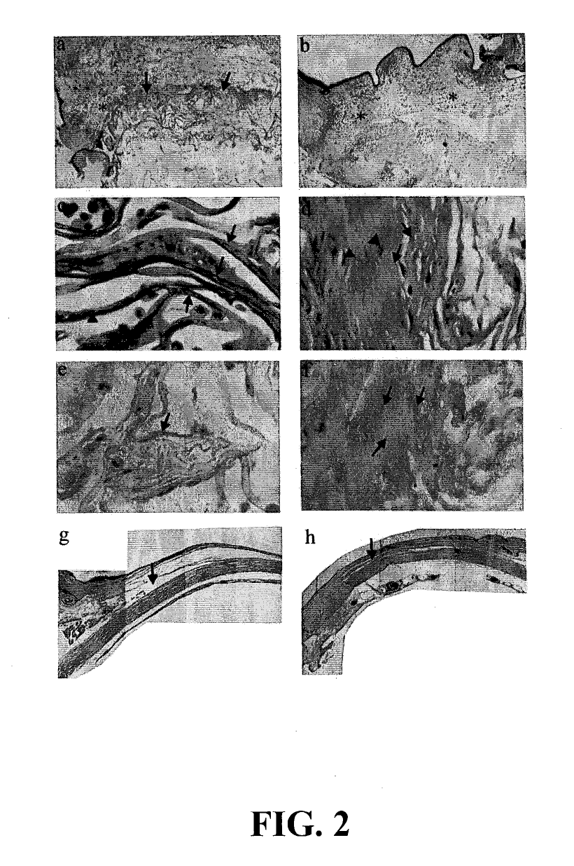

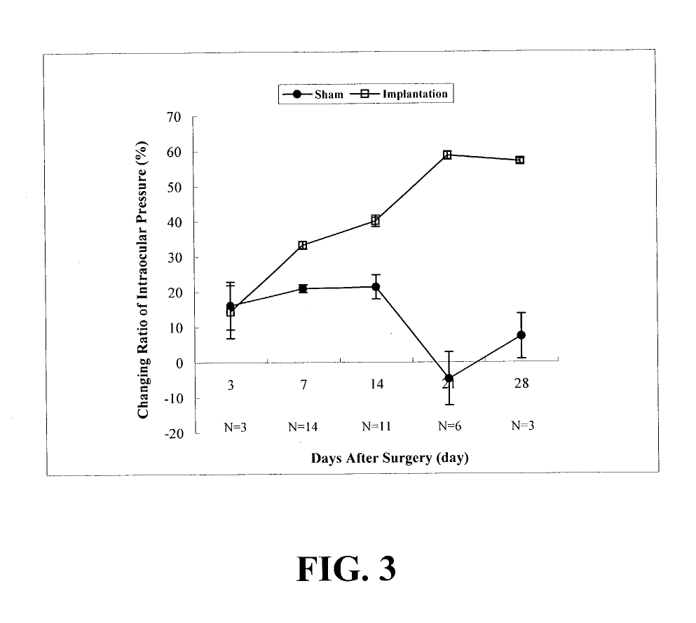

Wound areas of both implanted and un-implanted eyes evidence a typical acute inflammatory response at day 3 and day 7 after surgery. A mass of immunogenic cells aggregate, consisting of occasional elongated cells of fibroblasts, macrophages, and different types of lymphocytes. Collagens secreted by fibroblasts are deposited adjacently to the wound. The inflammatory cells and fibroblasts infiltrate into the area of the inner one third to one half of the scaffold adjacent to the sclera (FIG. 2a, 2b). Although the implanted scaffold is gradually degraded after 7 days, the remaining portion is visible. The remaining 3D porous structure for the regenerated cells distributes along the irregular pores. Fibroblasts predominantly extend beyond the pores and connect directly to the epithelium layer of the sclera. The immune responses have decreased gradually from day 14 and subside completely by day 21 after surgery. There is no difference in the immune response and in the subsiding time between the implanted and un-implanted wounds. The result indicates that the scaffold induced no additional immune response. Moreover, a loosely organized network is left with the invasion of scattered regenerated cells and secreted collagens on the implanted areas after the scaffold is degraded. Oppositely, the un-implanted surgery areas are occupied by a packed array of collagen fibers, and the conjunctiva of the un-implanted left eye is much thicker.

Identification of collagen by Masson's trichrome stain:

The tissue slides are deparaffinized in 100% xylene solution for 5 minutes (repeat 2 times) and rehydrated in 100%, 100%, 95%, 80%, 70% of ethanol in-and-out for 10 to 20 times. The tissue slides are mordanted in Bouin's Solution (Sigma M HT10-32) at 56° C. for one hour and then at room temperature overnight in a hood. Wash the tissue slides in running tap water to remove yellow color from tissue sections and rinse briefly in distilled water. Stain the tissue sections in Weigert's Iron Hematoxylin Solution (Sigma HT10-79) for 10 minutes. Wash in running tap water for 10 minutes and rinse in distilled water. Place the tissue slides in freshly prepared phosphomolybdic/phosphotungstic acid solution for 10-15 minutes. The fresh phosphomolybdic/phosphotungstic acid solution can be prepared by mixing phosphomolybdic acid (Sigma HT15-3) and 10% (w/v) phosphorungstic acid (Sigma HT15-2) in a 1:1 ratio by volume. Stain the tissue sections in Aniline Blue Solution for 5 minutes and rinse briefly in distilled water. Place the tissue slides in 1% glacial acetic acid solution for 3-5 minutes and dehydrate by sequential exposure to 70%, 80%, 90%, and 100% of ethanol for 10 seconds separately. After the secondary treatment in 100% ethanol, the tissue slides are transferred to 100% xylene solution for 10 seconds (repeat three times). Coverslip the tissue slides with Permount or Polymount, and observe under microscopy.

RESULTS

Stained collagen fibers appear in the implanted and un-implanted wound areas on day 3 after surgery. In tissue sections obtained from the 14th day after surgery, the scar forms in the un-implanted wound areas with a much more densely packed array of collagen fibers (FIG. 2c, 2d). The scar tissue continually develops up to day 28 after surgery (FIG. 2g, 2t). As compared with the results of immunostain of α-SMA on day 14 after surgery, there are many more myofibroblasts aligning compactly in the un-implanted wound areas (FIG. 2e, 2f). The observation confirms that the scaffold prevents scar formation.

Identify the Distribution of Active Myfibroblast by α-SMA Immunocylochemistry:

Deparaffin the tissue slides by heating at 56° C. for 10 minutes and dip the tissue slides into 100% xylene for 3 minutes (repeat 3 times). Transfer the tissue slides in 100% ethanol for 3 minutes (repeat 2 times) and expose sequentially to 90%, 80%, 70%, and 50% of ethanol for 3 minutes each step. Wash the tissue slides in 0.1 M PBS for 3 minutes (repeat 2 times), and place the tissue slides in 3% H₂O₂ at room temperature for 15 minutes. Wash the tissue slides in 0.1 M PBS containing with 0.2% Triton-X 100 (PBST) for 2-3 minutes (repeat 3 times). Block the non-specific bindings with 10% fetal bovine serum (FBS) in 0.1 M PBST at room temperature for 25 minutes. Incubate the tissue slides with α-SMA (Neomarkers) monoclonal antibody in a dilution of 1:500 at 4° C. overnight. After washing the tissue slides in PBST for 2-3 minutes (repeat 3 times), incubate the tissue slides with biotinylated anti-mouse/rabbit IgG (DAKO LSAB2^{R} system) in a dilution of 1:400 for 15 minutes at room temperature. Wash the tissue slides in PBST for 2-3 minutes (repeat 3 times). Drop streptavidin-HRP (DAKO LSAB2^{R} system) onto the tissue sections and incubate at room temperature for 15 minutes. Wash the tissue slides with PBST for 2-3 minutes (repeat 3 times). Conduct the chromogen (DAKO LSAB2^{R} system) reaction at room temperature for 10 minutes. Wash the tissue slides with PBST for 2-3 minutes (repeat 3 times). Counterstain with Hematoxylin solution for 30 seconds and wash in PBS for 3 minutes (repeat 3 times), followed by distilled water for 5 minutes (repeat 2 times). Cover the slides with glycerol gel (DAKO) at 56° C., and observe under microscopy.

RESULTS

In the unimplanted eye, immunostain of α-SMA reveals that numerous myofibroblasts aligned parallel to the sclera surface until day 14 after surgery, and the compactly aggregated collagen fibers secreted by myofibroblasts resulted in wound contraction. In contrast, only a few scattered myofibroblasts distributed in the implanted areas of the implanted eyes. They adhere randomly to the remaining scaffold and the wound area surroundings (FIG. 2e, 2f). As a result, wound contraction seldom happens in the implanted eyes. The wound contracts obviously on the day 21 after surgery because of the aggregation of collagen fibers in the subepithelial space and the contraction of the myofibroblasts adjacent to the wound of the un-implanted eyes. The subepithelial space is consequently smaller or collapsed. In comparison with the implanted eyes, the larger subepithelial space is due to the random distribution of collagen fibers and myofibroblasts as well as the degradation of collagen/C-6-S copolymers. Observation on day 28 after surgery shows that in implanted eyes the number of fibroblasts and myofibroblasts decreased and the stroma was replaced by the collagen fibers at the implanted wound areas. The collagen fibers align in a random orientation. In contrast, an obvious scar formation appears in the un-implanted eyes (FIG. 2g, 2h).

### EXAMPLE 5

### The Change of the Intraocular Pressure (IOP)

The intraocular pressure of the female New Zealand albino rabbits in Example 4 is measured with tonopen. Preceding measurement, the rabbits are anesthetized by an intramuscular injection with a half dosage of ketamine (35 mg/kg, BW) and xylazine (5 mg/kg, BW) before measurement on days 3, 7, 14, 21, and day 28. The same measurement is adopted before the rabbits are sacrificed for further morphological studies. Compared with the pressure before implantation, the changing rate of intraocular pressure is obtained by the formula below:

$$\begin{matrix}The IOP changing \\ rate ( \% )\end{matrix} = \frac{\begin{matrix}IOP before implantation - \\ IOP after implantation\end{matrix}}{IOP before implantation} \times 100 \%$$

RESULTS

In the un-implanted eyes, IOP decreases about 16% immediately after the channel connected to the anterior chamber is built and remains constant until 14 days, and then gradually increases, returning to the value measured before the surgery. In the situation of implanted eyes, IOP decreases about 14% immediately after the channel is built and then further decreases to 33% at day 7 after surgery. During tissue regeneration, the IOP decreases as well, and reaches to about 55% at day 28 after surgery (FIG. 3). The results temporally fit the morphological observation.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A drug-free biodegradable 3D porous scaffold comprising collagen and glycosaminoglycan.

2. The scaffold as claimed in claim 1, wherein the collagen comprises type I collagen.

3. The scaffold as claimed in claim 1, wherein the glucosaminoglycan comprises chondroitin-6-sulfate, chondrotin-4-sulfate, heparin, heparan sulfate, keratan sulfate, dermatan sulfate, chitin or chitosan.

4. The scaffold as claimed in claim 1, wherein collagen and glycosaminoglycan polymerize in forming CG copolymer.

5. The scaffold as claimed in claim 4, wherein the ratio of collagen and glycosaminoglycan in the polmerized CG copolymer is 10:1 in weight.

6. The scaffold as claimed in claim 1, wherein the scaffold comprises CG copolymer in a range of about 0.125% to 8%.

7. A method of making a drug-free biodegradable 3D porous collagen-glycosaminoglycan (CG copolymer) scaffold, comprising the steps of:
(a) dissolving collagen and glycosaminoglycan in 0.05 M acetic acid to form a solution;
(b) mixing the solution from step (a) at high speeds ranging from 3,500 rpm to 11,500 rpm;
(c) vacuuming and drying the mixed solution from step (b) until dry and in a sheet form, wherein final vacuum pressure is less than 30 mtorr and heating temperature is about 105° C.; and
(d) irradiating the dry sheet from step (c) with UV light for 2 hours on each face,
wherein the wavelength of the UV light is 254 nm.

8. The method as claimed in claim 7, wherein the collagen of the CG copolymer comprises type I collagen.

9. The method as claimed in claim 7, wherein the glycosaminoglycan of the CG copolymer comprises chondroitin-6-sulfate, chondrotin-4-sulfate, heparin, heparan sulfate, keratan sulfate, dermatan sulfate, chitin or chitosan.

10. The method as claimed in claim 7, wherein the ratio of collagen and glycosaminoglycan in the CG copolymer is 10:1 in weight.

11. The method as claimed in claim 7, wherein the scaffold comprises CG copolymer in a range of about 0.125% to 8%.

12. A method of modulating mammals' intraocular pressure on glaucoma, to cover hypotony, decrease incommodity, prevent scar formation and secondary infection after filtration surgery, using a drug-free biodegradable 3D porous scaffold comprising collagen and glycosaminoglycan, and the method comprising:
(a) cutting the scaffold into desired shape and size;
(b) immersing the cut scaffold from step (a) into a physiological buffer until saturated;
(c) dissecting a conjunctiva from a fornix to a limbus;
(d) exposing a sclera;
(e) building a channel over an intraocular trabeculum, whereby connecting an anterior chamber and a subconjuctival space;
(f) maintaining the scaffold from step (b) saturated with physiological buffer fluid before and during implantation;
(g) implanting the scaffold from step (b) into the subconjuctival space surrounding and above scleral flap, and including the channel connected between the anterior chamber and the subconjuctival space if necessary; and
(h) sewing incision resulted from step (c).

13. The method as claimed in claim 12, wherein the mammals comprise human beings.
